(19) United States
(12) Patent Application Publication
(10) Pub. No.: US 2023/0013733 A1
(43) Pub. Date: Jan. 19, 2023

(54) MICROCARRIERS WITH SCAFFOLD STRUCTURE AND CONTINUOUS OUTER WALL FOR CULTURING CELLS

(71) Applicant: Tantti Laboratory Inc., Taoyuan City (TW)

(72) Inventors: Pan LIN, Taoyuan City (TW); Tzu-Yuan CHAO, Taoyuan City (TW); Guan-Chi CHEN, Taoyuan City (TW); Yi-Fan HSIEH, Taoyuan City (TW)

(21) Appl. No.: 17/374,157

(22) Filed: Jul. 13, 2021

(51) Int. Cl. C12N 5/00; B29C 44/02; B29C 44/58

(52) U.S. Cl. CPC C12N 5/0075; B29C 44/02; B29C 44/58; C12N 2533/30; C12N 2533/54; B29K 2995/0056

(57) ABSTRACT

The invention relates to a microcarrier, comprising a continuous medium of a biocompatible polymer for culturing cells and having a three-dimensional scaffold architecture delineated peripherally by a continuous outer wall, in which spherical macropores are stacked to one another and interconnected by connecting pores. The continuous outer wall is formed with exposure pores at positions where it is in contact with the macropores, through which the interior of the microcarrier may be in fluid communication with the ambient culture medium. The microcarrier herein is produced by cast-molding and, therefore, has a continuous outer wall which provides additional mechanical strength while maintaining high porosity. The microcarrier thus produced is configured in the form of a basic geometrical body. The invention further relates to a cast-molding process for producing the microcarrier.

### BACKGROUND OF INVENTION

### 1. Field of the Invention

The present invention relates generally to a device for culturing cells, and more particularly to a microcarrier with a three-dimensional scaffold architecture for culturing cells. The microcarrier herein is formed with a continuous outer wall by using a cast-molding process, so as to maintain high porosity and good mechanical strength. The invention also relates to a cast-molding process for producing the microcarrier.

### 2. Description of Related Art

Traditionally, in vitro cell culture involves attaching cells onto plastic tissue culture vessels or extracellular matrix attachment proteins, and then giving appropriate liquid media to promote their growth and proliferation. However, the two-dimensional cell culture method is quite different from the actual physiological environment in vivo. It cannot simulate the interactions between cells and extracellular matrix and the interactions between cells in vivo, and it is not conducive to reproducing complex cell behaviors, such as cell migration, apoptosis, transcription regulation and receptor expression. Moreover, the two-dimensional cell culture only provides limited spaces for cell growth and is unfavorable to the mass production of cells. The three-dimensional cell culture technique is apparently a better solution in response to the industrial issue.

In the 1980s, Dr. Mina Bissell from the Lawrence Berkeley National Laboratory initiated the three-dimensional cell culture techniques in the research on breast cancer (see Petersen O. W., et al., PNAS, 89 (19): 9064-9068), which involve incubating cells with three-dimensional scaffolding biomaterials in vitro, allowing the cells to grow and migrate within the three-dimensional framework provided by the scaffolding biomaterials. In recent years, with the well-development of the manufacturing methods for cell culture scaffolds, three-dimensional cell culture technology has gradually replaced the traditional two-dimensional cell culture technology and has been widely used in biomedical fields, such as tissue engineering. In terms of applications, the three-dimensional scaffolds are useful in cell growth, tissue differentiation and remodeling in vitro and in vivo, and suitable for producing tissues for research or implantation. In terms of structures, they are formed with tiny pores stacked to one another, in which cells may be inoculated to grow and differentiate towards the predetermined three-dimensional directions, thereby producing regenerated tissues or organs.

U.S. Pat. No. 8,513,014 discloses a method for manufacturing blocks of three-dimensional scaffolding materials. The method involves using a bubble generator to introduce a gaseous flow into a continuous fluid to generate monodisperse bubbles, which are then gelled, disrupted under reduced pressure and cured into blocks with large sizes. U.S. Laid-Open Publication No. 2019/091690A1 teaches a microfluidic device with a T-junction for mass production of monodisperse bubbles, and U.S. Pat. No. 10,828,635 further teaches a device for isolating the bubbles from a liquid. The bubbles thus isolated were allowed to self-assemble into a close-packed arrangement and then gelled, disrupted under reduced pressure and cured into bulky blocks of three-dimensional scaffolding materials. U.S. patent application Ser. No. 17/184,276 describes a method of producing three-dimensional scaffolding materials using the high internal phase emulsion (HIPE) templating technology. Other conventional processes for preparing scaffolding materials include salting-out process, freeze drying process and solid freeform production process.

While the conventional methods described above have been able to produce three-dimensional scaffolds in the form of bulky blocks, it is still a practical concern that the fresh culture medium and the metabolites should be able to easily diffuse into and out of the three-dimensional scaffolds throughout cell culture, thereby facilitating healthy growth of the cells attached to the scaffolds. Therefore, the block materials must be subdivided into finely ground microcarriers before use, allowing substances to pass in and out of the microcarriers. As per the practitioners' experiences, the appropriate size of a microcarrier is from 500 μm to 3,000 μm, depending upon the types of cells to be cultured and the culture conditions. The microcarriers with this size would remain suspended in the culture medium under continuous stirring.

The dividing of block materials into microcarriers generally involves using a conventional mechanical high-speed cutting process. However, the excessive mechanical stress imposed on the scaffolding materials during the cutting would cause considerable structural defects in microcarriers, giving them a broken and irregular appearance. The microcarriers thus produced contain spherical macropores completely exposed to the ambient and do not have a continuous and smooth outer wall. The microcarriers were proved to be fragile and easily collapsed under continuous agitation during cell culture due to insufficient mechanical strength. Their overly exposed porous structure further makes the attached cells suffer from excessive shear force generated by the flowing culture medium, resulting in low cell productivity. Moreover, the size distribution of the microcarriers obtained by high-speed cutting depends on how they are cut. If high dimensional uniformity is required, a relatively complicated cutting process and a longer processing time are needed. Alternatively, a simplified cutting process with a shortened processing time would normally lead to a broad size distribution from several microns to thousands of microns. Microcarriers produced in this way have to be sieved to certain particle sizes suitable for cell culture (with a ratio of maximum size to minimum size being no more than 1.5), the yield of which is extremely low as per practitioners' experiences. Although ultra-short wavelength laser radiation may be used to carry out the cutting process, users would still have to face the disadvantages of complicated processing procedures, too many pores exposed from product's surfaces and expensive processing equipment. In addition, laser cutting would normally produce a kerf of around 0.1 mm. Assuming that laser cutting is used to produce microcarriers with a size of 1 mm³, the production yield of the microcarriers is estimated to be (1/1.1)³=75%, which means a yield loss of 25%. Therefore, there is still an eager need in the related technical field for highly porous microcarriers with uncompromised mechanical strength, as well as cost- and time-effective methods for producing microcarriers with high production yield.

### SUMMARY OF THE INVENTION

The present invention discloses a microcarrier having high porosity, good mechanical strength and a three-dimensional scaffold architecture, all of which make it ideal for cell culture. The invention further relates to a novel process for producing the microcarrier. The microcarrier herein is produced by a cast-molding process, which involves filling a foam of a biocompatible polymer into an appropriate mold, followed by disrupting the bubbles and curing the foam, and then removing the microcarrier from the mold. The foam is substantially composed of bubbles dispersed and stacked in a solution of the biocompatible polymer. After the foam is cured, the polymer solution is solidified into a continuous medium with a three-dimensional scaffold architecture, while the original space where the bubbles occupy becomes spherical macropores. Adjacent macropores are interconnected through connecting pores at the contact points thereof due to the disruption of the bubbles. When the foam is filled into the mold, it contacts the mold surface and is exposed to the ambient air through the mold pores, resulting in spontaneous formation of a thin layer of the polymer solution which envelops the foam body due to the surface tension of the polymer solution. The thin layer of polymer solution is converted into a continuous outer wall of the microcarrier after curing. Exposure pores are formed on the continuous outer wall during bubble disruption at positions where some of the spherical macropores are in contact with the continuous outer wall.

Therefore, in the primary aspect provided herein is a microcarrier with a size from 500 μm to 3,000 μm, depending upon the types of cells to be cultured and the culture conditions. The overall profile of the microcarrier exhibits in the form of a simple basic geometrical body. It has a three-dimensional scaffold architecture composed of the continuous medium described above, which is characterized by the structural features of multiple spherical macropores stacked one another, connecting pores interconnecting the macropores, as well as exposure pores and a continuous outer wall. The respective spherical macropores are interconnected with adjacent macropores through connecting pores. The continuous outer wall is formed with exposure pores at positions where it is in contact with the macropores, through which the interior of the microcarrier may be in fluid communication with the ambient culture medium.

The spherical macropores, connecting pores and exposure pores constitute sinuous channels in the microcarrier, through which the culture medium and metabolites may diffuse. These structure features may also create diffusion bottlenecks which would slow down the substance diffusion in the microcarrier and shall be avoided to ensure that the efficiency of substance diffusion satisfies the needs of the cells growing in the microcarrier. Based on the empirical results obtained by the inventors, if the diameter of a spherical macropore is regarded as 1 unit, then the suitable diffusion distance for substances would be from about 3 units to about 5 units. Therefore, the ratio of the characteristic dimension of the microcarrier to the diameter of the spherical macropore is from about 6:1 to about 10:1.

In another aspect provided herein is a method for producing the microcarrier described above, which comprises the steps of:

A. preparing a polymeric foam containing a continuous phase and a dispersed phase immiscible with the continuous phase and composed of mutually separated units dispersed in the continuous phase, wherein the continuous phase comprises a component selected from the group consisting of a biocompatible polymer, a monomer thereof, an oligomer thereof and a combination thereof;

B. filling the polymeric foam into a porous plate mold, and curing the polymeric foam to obtain a continuous medium, wherein the porous plate mold defines a plurality of micro-through holes connecting two main surfaces of the porous plate mold, and each of the micro-through holes is configured in the form of a basic geometrical body with a characteristic dimension from 500 μm to 3,000 μm, and wherein the respective mutually separated units in the dispersed phase have a diameter which has a ratio from about 1:6 to about 1:10 to the characteristic dimension; and

C. releasing the continuous medium from the porous plate mold to obtain a microcarrier with a three-dimensional scaffold structure and a continuous outer wall for culturing cells.

In a preferred embodiment, the respective exposure pores of the microcarrier have a diameter which is substantially smaller than that of the spherical macropore adjacent thereto.

In a preferred embodiment, at least 50% of the spherical macropores in the microcarrier are in a close-packing arrangement.

In a preferred embodiment, the basic geometrical body is selected from the group consisting of a cylinder, a sphere, a cone, a cube, a cuboid, a prism and a pyramid. In a more preferred embodiment, the basic geometrical body is selected from a cylinder.

In a preferred embodiment, the biocompatible polymer is selected from the group consisting of proteins, polysaccharides, synthetic polymers and a combination thereof. In a more preferred embodiment, the biocompatible polymer is selected from the group consisting of gelatin, collagen, fibrins, agarose, hyaluronic acid, chitin, alginates, celluloses and gellan gum.

The microcarrier according to invention, as well as the manufacturing process thereof, can bring about the following advantageous effects:

1. The advantageous effects resulted from the internal porous architecture of the microcarrier:

The spherical macropores of the microcarrier herein are interconnected with one another, forming a continuous porous network structure with interconnected pores. The microcarrier has an extremely large specific surface area and, therefore, is suitable for cells to enter the spherical macropores and to adhere to and grow on the walls of the macropores. Fresh culture medium and metabolites can diffuse rapidly between the interior of the microcarrier and the ambient through the connecting pores and exposure pores, thereby promoting cell growth.

2. The advantageous effects resulted from the continuous outer wall of the microcarrier:

The turbulent flow generated by continuous agitation of the culture medium during cell culture will cause frequent impacts on the scaffolds and/or cells. The substantially smooth continuous outer wall provides protection for the internal scaffolding architecture and cells, thus reducing the damages from the turbulent flow. The continuous outer wall is structurally integrated with, and therefore helps maintaining the integrity of, the internal scaffolding architecture in the microcarrier. The continuous outer wall also contributes to uniformly disperse the shear stress generated by the ambient culture medium. As a result, the mechanical strength of the three-dimensional scaffold is so enhanced as to prevent the microcarrier from collapsing under cell culture conditions.

3. The advantageous effects resulted from the simplified cast-molding process:

As described above, after filled into an appropriate mold, the foam is processed through bubble disruption, curing and mold-releasing procedures to obtain a microcarrier ready for use in cell culture. The process herein may exclude the following steps and the drawbacks resulted therefrom: (1) a preliminary process for manufacturing bulky blocks of three-dimensional scaffolding materials; (2) a time-consuming and cost-ineffective mechanical or laser cutting process; (3) a high kerf loss resulted from the cutting process; and (4) surface defects of the microcarrier resulted from the cutting process, as well as the adverse effects caused thereby, such as low mechanical strength and poor production yield of cells. Therefore, the invention herein can increase greatly the productivity.

4. Systematic advantageous effects resulted from of the cast-molding process:

The mold employed in the cast-molding process can be optimized in terms of shape and size and formed with a vast amount of micro-through holes, so that the microcarriers can be produced in massive amount with a narrow size distribution, thereby facilitating controllability, predictability and analyzability of the cell culture.

### BRIEF DESCRIPTION OF THE DRAWINGS

FIG. 1 is a schematic cross-sectional diagram of a cylindrical-shaped microcarrier according to one embodiment of the invention, wherein the continuous medium and the continuous outer wall are integrally formed as a monolithic piece after the polymeric foam is cured, though they are described as two components below for the sake of illustration;

FIG. 2A is an electron microscopic image of the cylindrical-shaped microcarrier prepared according to one embodiment of the invention, showing the top surface of the microcarrier, and FIG. 2B is a schematic diagram of the microcarrier;

FIG. 3A is another electron microscopic image of the cylindrical-shaped microcarrier prepared according to one embodiment of the invention, showing the lateral surface of the microcarrier, and FIG. 3B is a schematic diagram of the microcarrier;

FIG. 4A and FIG. 4B are another electron microscopic images of the microcarrier prepared according to one embodiment of the invention, showing the porous architecture of the microcarrier;

FIG. 5 is the flow chart of the inventive method for producing the microcarrier;

FIG. 6 is an optical microscopic image according to one embodiment of the invention, showing the lyophilized foam in the mold; and

FIG. 7 is an electron microscopic image of the microcarrier prepared by a conventional cutting process.

### DETAILED DESCRIPTION OF THE INVENTION

Unless specified otherwise, the following terms as used in the specification and appended claims are given the following definitions. It should be noted that the indefinite article “a” or “an” as used in the specification and claims is intended to mean one or more than one, such as “at least one,” “at least two,” or “at least three,” and does not merely refer to a singular one. In addition, the terms “comprising/comprises,” “including/includes” and “having/has” as used in the claims are open languages and do not exclude unrecited elements. The term “or” generally covers “and/or”, unless otherwise specified. The terms “about” and “substantially” used throughout the specification and appended claims are used to describe and account for small fluctuations or slight changes that do not materially affect the nature of the invention.

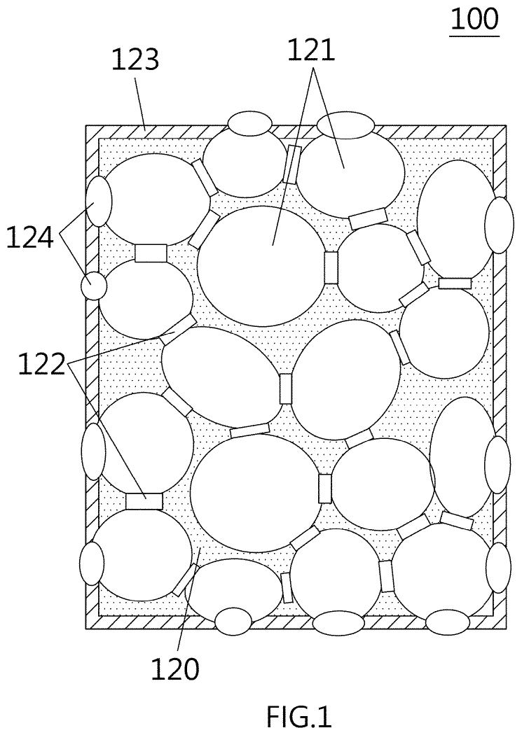

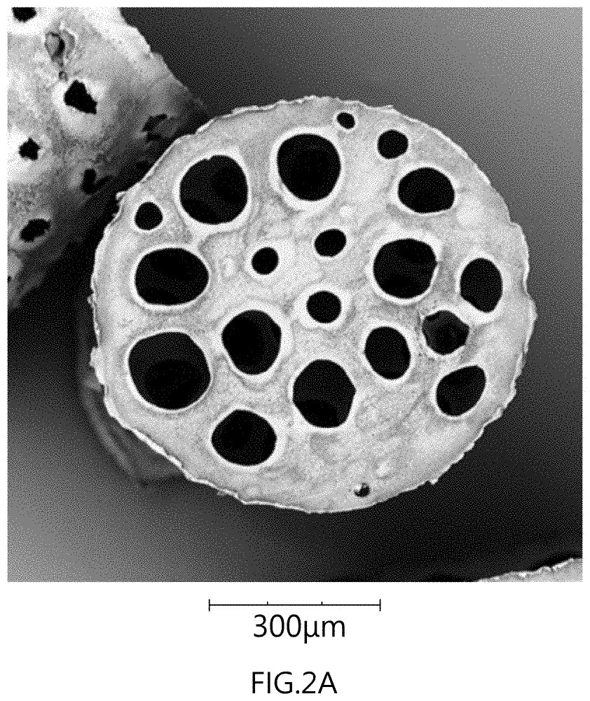

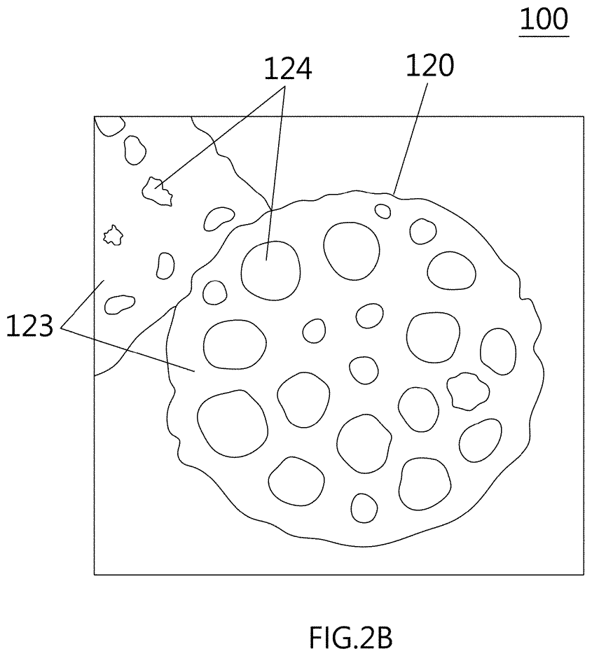

FIG. 1 is a schematic diagram of an embodiment of the invention, showing that a microcarrier 100 mainly comprises a continuous medium 120. The term “continuous medium” as used herein may refer to a monolithic solid material made of one or more biocompatible polymers, on which cells may attach, grow, proliferate and migrate. The term “biocompatible” is used herein to describe materials which do not induce adverse effects on biosystems, such as cells, tissues and organs. Suitable biocompatible polymers for producing microcarriers are known in the related art, which include, but are not limited to, proteins, e.g., gelatin, collagen and fibrins; polysaccharides, e.g., agarose, hyaluronic acid, chitin, alginate, cellulose and gellan gum; synthetic polymers, such as biodegradable polymers, e.g., polyester-amides, polycaprolactone (PCL), polylactic acid (PLA), polyglycolic acid (PGA) and poly(lactic-co-glycolic acid) (PLGA), and non-biodegradable polymers, e.g., polydimethylsiloxane (PDMS), thermoplastic polyurethanes, polyethylene terephthalate (PET), polytetrafluoroethylene (PTFE), polyvinylidene difluoride (PVDF) and polystyrenes; and combinations thereof. In the preferred embodiments shown in FIGS. 2A-2B, FIGS. 3A-3B, FIGS. 4A-4B and FIG. 6, the microcarrier 100 is made of collagen.

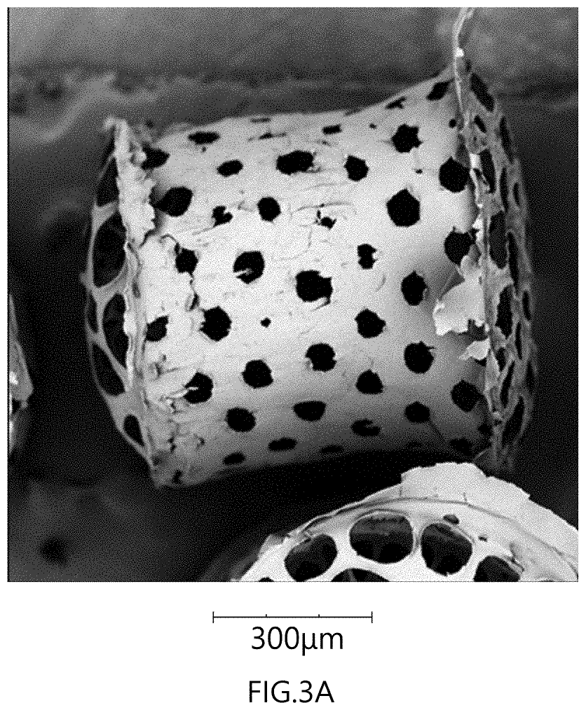

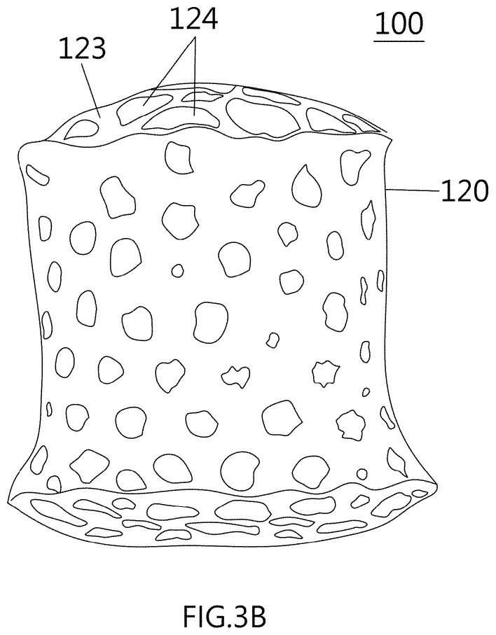

As shown in FIGS. 1, 2A-2B and 3A-3B, the microcarrier 100 is substantially in the configuration of a basic geometrical body. The term “basic geometrical body” herein may refer to a three-dimensional structure composed of simple curved surfaces and/or planar surfaces, which comprises geometrical bodies with a curved surface, such as a cylinder, a sphere and a cone, and planar geometrical bodies, such as a cube, a cuboid, a prism and a pyramid. The microcarrier 100 can be fabricated into any configuration of the basic geometrical body by cast-molding, so long as it can be manipulated conveniently by the cast-molding and mold-releasing procedures. In preferred embodiments, the microcarrier 100 is configured into a cylinder shape. As stated below, the microcarriers 100 are produced by cast-molding in a mold of a fixed uniform dimension, so as to have a narrow size distribution. According to the invention, the microcarrier 100 has a characteristic dimension from 500 μm to 3,000 μm, depending upon the types of cells to be cultured and the culture conditions. The term “characteristic dimension” as used herein may refer to the largest dimension of the outer profile of the microcarrier 100, such as the length, width, height or diameter of the microcarrier 100. For example, the characteristic dimension of a cylindrical microcarrier may refer to the height or diameter of the microcarrier. Herein, the term “characteristic dimension” is also intended to describe the largest dimension of the micro-through holes formed in the mold, such as the diameter and height of the micro-through holes. Therefore, the dimensions of the micro-through holes of the mold are predetermined according to the invention, such that the microcarriers 100 are fabricated to have a characteristic dimension from 500 μm to 3,000 μm, allowing them to be suspended in a liquid cell culture medium. In one embodiment, the microcarrier 100 has a characteristic dimension from 500 μm to 880 μm, so that the characteristic dimension of the microcarrier 100 is smaller than the caliber of industrial and laboratory pipette tips, allowing the microcarrier 100 to be sampled along with a predetermined volume of a cell culture sample through the conventional pipettes, thereby facilitating rapid monitoring of cell growth and quick adjustment of culture conditions. The characteristic dimension of the microcarrier 100 may be measured through electron microscopy, as shown in FIG. 3A. The characteristic dimension may also be measured by sieving. That is to say, the microcarrier 100 can pass through a 6-mesh screen (with openings of 3,350 μm) based on the Tyler standard screen scale and can even pass through an 18-mesh screen (with openings of 880 μm), but it cannot pass through a 32-mesh screen mesh (with openings of 500 μm).

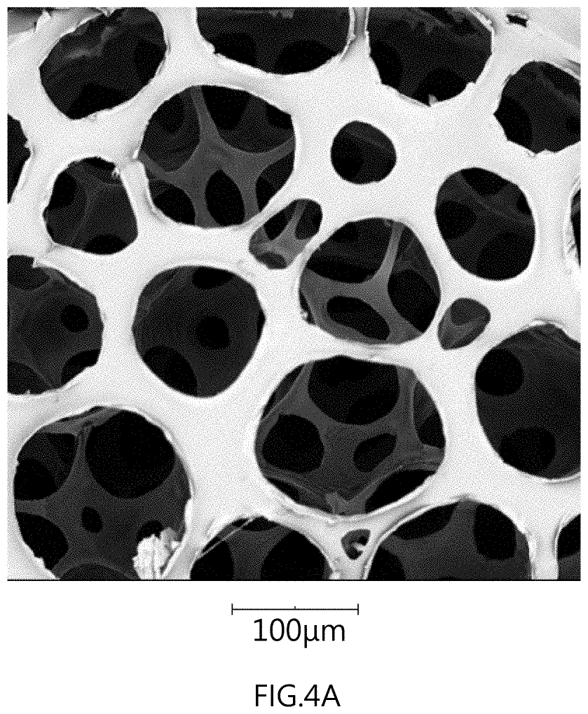

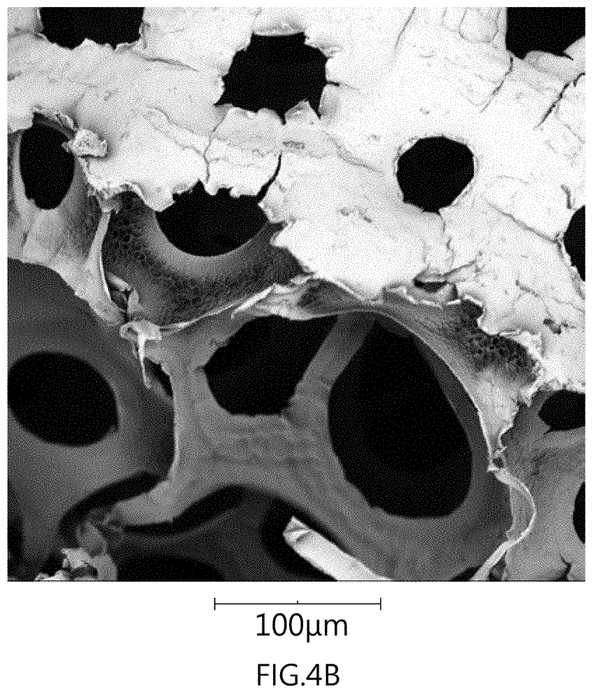

As shown in FIGS. 1 and 4A-4B, the microcarrier 100 is formed inside with spherical macropores 121. As stated below, the sizes of mutually separated units in the dispersed phase (e.g., bubbles or droplets) may be adjusted by controlling the parameters and conditions of the step for preparing the polymeric foam, so that the respective spherical macropore 121 have a diameter which has a ratio from about 1:6 to about 1:10 to the characteristic dimension of the microcarrier 100, thereby facilitating the substance diffusion between the interior of the microcarrier 100 and the ambient. For example, the microcarrier having a characteristic dimension of about 1,000 μm is formed with spherical macropores having a diameter ranging from 100 μm to 170 μm, whereas the microcarrier having a characteristic dimension of about 3,000 μm is formed with spherical macropores having a diameter ranging from 300 μm to 500 μm. In one embodiment, the spherical macropores 121 is fabricated to have a diameter from 5 μm to 500 μm and, preferably, have a diameter from 50 μm to 200 μm. The spherical macropores 121 are arranged to be adjacent to one another. The term “adjacent to” as used herein means that the respective spherical macropores in the microcarrier are normally interconnected with at least one other spherical macropore via at least one connecting pore. The sizes of the connecting pores 122 can be controlled by adjusting the contact areas between the mutually separated units in the dispersed phase (e.g., bubbles or droplets) during the manufacturing of the polymeric foam. In some embodiments, all the spherical macropores 121 have a substantially uniform diameter, whereas in the other embodiments, spherical macropores 121 has a relatively wide size distribution, depending on whether the bubbles or droplets are produced to be monodisperse or polydisperse. In one embodiment, at least some spherical macropores 121 in the microcarrier 100 are orderly arranged, such as in a close-packing arrangement, i.e., in a hexagonal closest packing (hcp) arrangement, a face centered cubic packing (fcc) arrangement, or a combined arrangement thereof. Preferably, at least 50% of the spherical macropores 121, more preferably at least 60% of the spherical macropores 121, and most preferably at least 70% of the spherical macropores 121, such as at least 80% of the spherical macropores 121, in the microcarrier 100 are in a close-packing arrangement.

The microcarrier 100 comprises a continuous outer wall 123. For example, in the case where the microcarrier 100 is fabricated into a cylindrical configuration, the continuous outer wall 123 includes a top surface, a bottom surface and a lateral curved surface, as shown in FIGS. 1, 2A-2B and 3A-3B. The term “continuous outer wall” as used herein means that all points on the outer wall are directly connected without interruption. The continuous outer wall 123 helps maintaining the structural integrity of the continuous medium 120 and enhances the mechanical strength of the microcarrier 100, preventing it from collapsing under cell culture conditions. Owing to the presence of the continuous outer wall 123, the spherical macropores 121 are not completely exposed to the ambient, and the cells attached to the microcarrier 100 are protected against the shear force generated by the flowing culture medium under cell culture conditions, thereby providing a stable environment for cells to grow and proliferate.

As shown in FIGS. 1, 2A-2B and 3A-3B, the continuous outer wall 123 is formed with a plurality of exposure pores 124 communicating the interior with the exterior of the microcarrier 100, and some of the spherical macropores 121 are partially exposed to the ambient through the exposure pores 124. That is to say, every exposure pore 124 has a diameter substantially smaller than the diameter of the spherical macropore 121 adjacent thereto, so that the spherical macropore 121 will not be completely exposed to the ambient. The cells may enter the microcarrier 100 through the exposure pores 124.

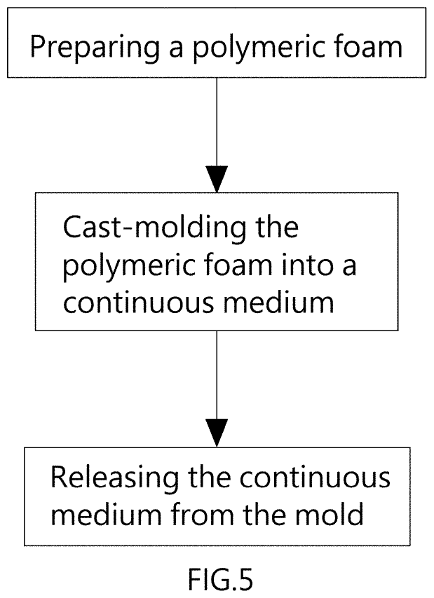

FIG. 5 shows the flow chart of the process for producing the microcarrier according to the invention, which comprises Step A: preparing a polymeric foam; Step B: cast-molding the polymeric foam into a continuous medium; and Step C: releasing the continuous medium from the mold.

Step A involves preparing a polymeric foam, which comprises a continuous phase, and a dispersed phase immiscible with the continuous phase and composed of mutually separated units dispersed in the continuous phase. According to the invention, the continuous phase is the phase which is to be cured into a continuous medium and comprises a component selected from the group consisting of the biocompatible polymer described above, a monomer thereof, an oligomer thereof and a combination thereof. The continuous phase may further comprise other components necessary for the curing, such as a crosslinking agent, a polymerization initiator, an emulsification stabilizer, a surfactant, a salt, a solvent and so on. The continuous phase is usually in the form of a viscous fluid at room temperature. The term “cure” or “curing” as used herein may refer to a process of subjecting the fluidic continuous phase to a physical and/or chemical bridging treatment, thereby converting it into a continuous medium with a stable solid configuration. In some embodiments, the dispersed phase is a gas, and the continuous phase is an oily or aqueous solution or suspension. In the other embodiments, the dispersed phase is an aqueous solution, and the polymeric foam is in the form of a water-in-oil emulsion.

Processes for producing the polymeric foam above can be seen in, for example, U.S. Pat. No. 8,513,014 and U.S Laid-open Publication No. 2019/091690A1, both of which involve using multiphase flow processing to introduce a gaseous flow or a liquid flow into a continuous phase through microfluidic devices. By virtue of the specially designed microfluidic devices disclosed therein, as well as proper management of the fluid flow rates, bubbles or droplets are evenly dispersed in the continuous phase. Based on the processes disclosed therein, monodisperse bubbles and droplets may be produced in large quantity by adjusting the size and geometric shape of the microfluidic passageway and/or the properties (such as viscosity and surface tension) and flow rates of the fluids. The monodisperse bubbles or droplets thus produced may be further arranged in a close-packing arrangement, so as to produce microcarriers formed with spherical macropores having a uniform diameter. The patent and patent publication above are herein incorporated by reference in their entirety.

Another process for foam production involves vigorously agitating a continuous phase composition and an immiscible dispersed phase composition by a high-speed homogenizer, so that the dispersed phase is evenly dispersed in the continuous phase to obtain a water-in-oil emulsion. Optionally, the water-in-oil emulsion may be subjected to a forced sedimentation, thereby increasing the volume fraction of the dispersed phase relative to the continuous phase in the emulsion to obtain a high internal phase emulsion (HIPE). The microcarriers produced from the HIPE would have an increased porosity and be formed with enlarged connecting pores. It is known by a person skilled in the art that the size and uniformity of the bubbles/droplets in the dispersed phase can be adjusted by changing the volume ratio of the dispersed phase to the continuous phase in the emulsion and/or by adjusting the agitation speed and temperature.

Other methods for producing polymeric foams are also applicable to the invention.

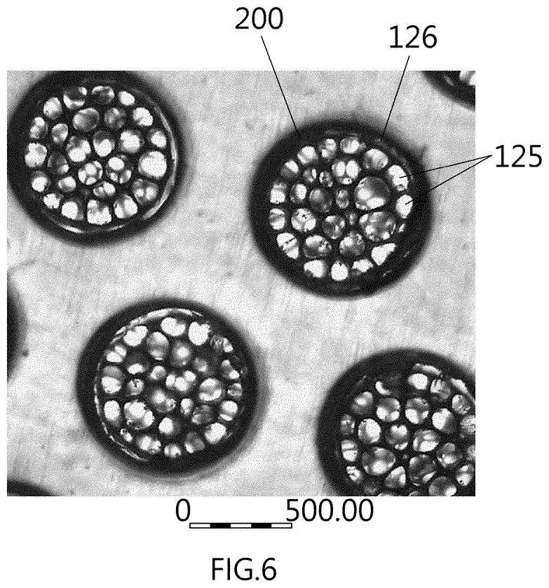

In Step B, a porous plate mold is prepared, which defines a plurality of micro-through holes connecting two main surfaces of the mold. Preferably, these micro-through holes arranged in an array. The micro-through holes are fabricated in the configuration of a basic geometrical body. That is to say, they have a configuration selected from the group consisting of a cylinder, a sphere, a cone, a cube, a cuboid, a prism and a pyramid. In the preferred embodiment shown in FIG. 6, the micro-through holes are configured into a cylinder shape. The micro-through holes have a characteristic dimension from 500 μm to 3,000 μm, which is to say, the heights of the respective micro-through holes (equal to the thickness of the porous plate mold) are from 500 μm to 3,000 μm, and/or the diameters of the respective micro-through holes are from 500 μm to 3,000 μm. In one embodiment, the micro-through holes are so fabricated as to have a characteristic dimension from 500 μm to 880 μm. The porous plate mold may be made of any inert material which is chemically unreactive to the polymeric foam, and examples of the inert material include carbon fiber, ceramics, glass, silica, plastic materials, e.g., polyvinyl chloride (PVC), polyoxymethylene (POM), polycarbonate (PC), polyphenylene oxide (PPO), PA6/66 nylon, polycarbonate (PC)/acrylonitrile butadiene styrene (ABS) composites, polyethylene terephthalate (PET), polyetherimide (PEI), polymethyl methacrylate (PMMA), polyphenylene sulfide (PPS), polyethylene (PE), polypropylene (PP), polystyrene (PS) and ethylene vinyl acetate (EVA), and metal materials, e.g., stainless steel, Ti, Al and Al—Mg alloys.

According to the invention, the diameters of the mutually separated units in the dispersed phase of the foam are adjusted to have a ratio from about 1:6 to about 1:10 to the characteristic dimension of the micro-through holes. In one embodiment, the diameters of mutually separated units are from 5 μm to 500 μm and, preferably, from 50 μm to 200 μm.

The process of fabricating the porous plate mold is well known by a person skilled in the art and may be modified depending on the material used. For example, when the mold is made of plastic material, useful manufacturing processes may include, but be not limited to, injection molding, cast-molding and thermoforming, followed by a conventional punching or drilling process to create the micro-through holes. When the mold is made of metal material, it may be fabricated by using conventional metal processing methods, such as stamping, rolling, cast-molding and forging, optionally followed by a conventional punching or drilling process to create the micro-through holes.

The polymeric foam prepared in Step A is poured on the porous plate mold, and then the mold surface is scraped with a plastic scraper at a proper rate, so that the foam is extruded to fill up the micro-through holes. If there is foam spilling out of the lower part of the mold, it could be removed by using the scraper. In light of its low density, the foam exhibits sufficient adhesive force to the inner walls of the micro-through holes for keeping itself within the holes, so that it will not drain before being cured. Again, the term “cure” or “curing” herein may refer to a process of subjecting the fluidic continuous phase to a physical and/or chemical bridging treatment, thereby converting it into a continuous medium with a stable solid configuration. The reaction conditions of the curing may vary depending on the type of the biocompatible polymer used, but they are well known in the art. For example, in the embodiment where collagen or gelatin is used as the biocompatible polymer, the foam filled in the mold is dehydrated at a low temperature, so as to be gelled into solid. In the embodiment where alginate serves as the biocompatible polymer, the foam may be added with a solution of a divalent metal ion, such as calcium ion and magnesium ion, to induce cross-linking between alginate molecules, so that the foam is gelled into solid. In the embodiment where polystyrene is used as the biocompatible polymer, free-radical polymerization of the styrene monomers in the continuous phase may be carried out to cure the foam. The cured continuous medium may be further lyophilized, preferably under vacuum, to facilitate disruption of the bubbles or droplets in the dispersed phase to generate connecting pores and exposure pores.

FIG. 6 shows a lyophilized collagen foam within a micro-through hole of a mold 200, the empty space left after removal of the dispersed phase 125 will become spherical macropores in the continuous medium. It is important to note that as shown in FIG. 6, a thin layer of the continuous phase liquid 126 remains between the dispersed phase 125 and the mold 200 due to the cohesion of the continuous phase liquid 126, and this thin layer is converted into the continuous outer wall 123 after curing. The continuous outer wall 123 thus formed is complementary in shape to the micro-through hole of the mold 200, so as to help maintaining the structural integrity of the continuous medium 120 and enhances the mechanical strength of the microcarrier 100, thereby preventing the microcarrier 100 from collapsing under cell culture conditions. During lyophilization, the vulnerable interfaces between the respective spherical macropores and the continuous outer wall 123 are disrupted due to the unbalance between internal and external pressures of the continuous medium 120, thereby forming the exposure pores. The respective exposure pores have a diameter which is substantially smaller than the diameter of the spherical macropore adjacent thereto, so that the spherical macropores will not be fully exposed. Sponge-like or honeycomb-like continuous media are obtained after curing, which comprise multiple spherical macropores adapted for cell attachment and growth.

Step C may comprise any process which is useful in releasing the continuous medium 120 from the mold without causing substantial structural damage to it. For example, the continuous medium 120 may be blown out from the mold by compressed air. In the embodiment where collagen or gelatin is used as the biocompatible polymer, the continuous medium 120 released from the mold may be dried and thermally bridged at a temperature higher than 37° C. For example, the continuous medium 120 made of collagen may be placed in an oven (DENG YNG D060) and dried at 50° C. under vacuum for 1 hour, and then baked at 150° C. for 12-48 hours to obtain a microcarrier 100. In step C, a large batch of microcarriers 100 which have a narrow size distribution and are formed with a continuous outer wall may be harvested.

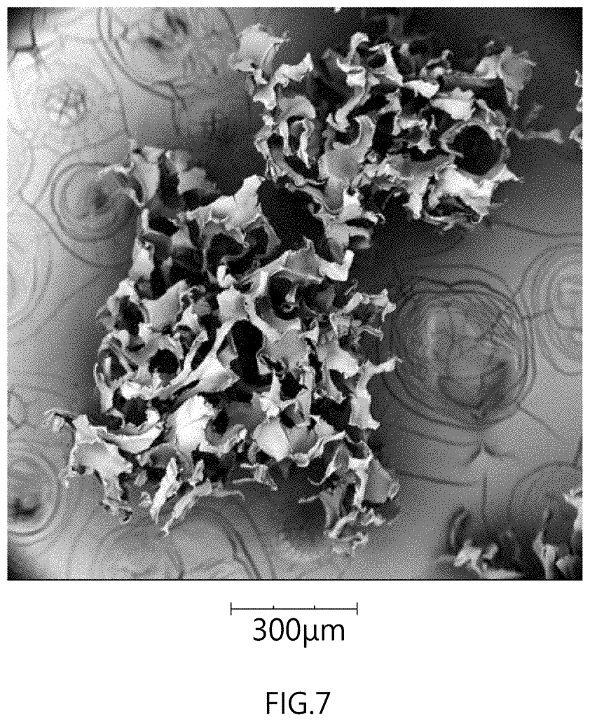

FIG. 7 shows the conventional microcarriers prepared through a traditional cutting process, which have broken profiles with irregular sizes. In contrast, the microcarriers according to the invention, as shown in FIGS. 2A-2B and 3A-3B, have a basic geometrical body configuration with structural integrity, while exhibiting a narrow size distribution and an excellent mechanical strength. According to the empirical tests conducted under actual cell culture conditions, the microcarriers according to the invention were not disintegrated or collapsed under a 14-day agitation in a stirring bioreactor.

The microcarrier herein may be extensively used in various technical fields, such as tissue engineering, oncology, regenerative medicine, drug screening test and stem cell biology. Taking advantage of its high mechanical strength, high specific surface area and high porosity, the microcarrier herein is adapted for incubation with different types of cells in vitro to mass-produce the cells, or for implantation with cells in vivo to remodel damaged tissues. In the embodiments where proteins or polysaccharides are used as scaffolding material, appropriate enzymes, such as trypsin, may be used to dissolve the microcarriers to recover cells.

While the invention has been described with reference to the preferred embodiments above, it should be recognized that the preferred embodiments are given for the purpose of illustration only and are not intended to limit the scope of the present invention and that various modifications and changes, which will be apparent to those skilled in the relevant art, may be made without departing from the spirit and scope of the invention.

We claim:

1. A microcarrier with a three-dimensional scaffold architecture for culturing cells, comprising:
a continuous medium made of a biocompatible polymer, which is substantially in the configuration of a basic geometrical body and has a characteristic dimension from 500 μm to 3,000 μm;
wherein said microcarrier is formed with a plurality of spherical macropores arranged to be adjacent to one another, with the spherical macropores being interconnected through connecting pores, wherein the respective spherical macropores have a diameter which has a ratio from about 1:6 to about 1:10 to the characteristic dimension; and
wherein the microcarrier has a continuous outer wall, which is formed with exposure pores at positions where it is in contact with the spherical macropores.

2. The microcarrier according to claim 1, wherein the respective exposure pores of the microcarrier have a diameter which is substantially smaller than that of the spherical macropore adjacent thereto.

3. The microcarrier according to claim 2, wherein the basic geometrical body is selected from the group consisting of a cylinder, a sphere, a cone, a cube, a cuboid, a prism and a pyramid.

4. The microcarrier according to claim 3, wherein the continuous medium has a characteristic dimension from 500 μm to 880 μm.

5. The microcarrier according to claim 4, wherein the biocompatible polymer is selected from the group consisting of proteins, polysaccharides, synthetic polymers and a combination thereof.

6. The microcarrier according to claim 5, wherein the biocompatible polymer is selected from the group consisting of gelatin, collagen, fibrins, agarose, hyaluronic acid, chitin, alginates, celluloses and gellan gum.

7. The microcarrier according to claim 6, wherein the spherical macropores have a diameter from 50 μm to 200 μm.

8. The microcarrier according to claim 7, wherein at least 50% of the spherical macropores in the microcarrier are in a close-packing arrangement.

9. A method for producing a microcarrier, comprising the steps of:
A. preparing a polymeric foam containing a continuous phase and a dispersed phase immiscible with the continuous phase and composed of mutually separated units dispersed in the continuous phase, wherein the continuous phase comprises a component selected from the group consisting of a biocompatible polymer, a monomer thereof, an oligomer thereof and a combination thereof;
B. filling the polymeric foam into a porous plate mold, and curing the polymeric foam to obtain a continuous medium, wherein the porous plate mold defines a plurality of micro-through holes connecting two main surfaces of the porous plate mold, and each of the micro-through holes is configured in the form of a basic geometrical body with a characteristic dimension from 500 μm to 3,000 μm, and wherein the respective mutually separated units in the dispersed phase have a diameter which has a ratio from about 1:6 to about 1:10 to the characteristic dimension; and
C. releasing the continuous medium from the porous plate mold to obtain a microcarrier with a three-dimensional scaffold structure and a continuous outer wall for culturing cells.

10. The method for producing microcarriers according to claim 9, wherein the basic geometrical body is selected from the group consisting of a cylinder, a sphere, a cone, a cube, a cuboid, a prism and a pyramid.

11. The method for producing microcarriers according to claim 10, wherein the continuous medium has a characteristic dimension from 500 μm to 880 μm.

12. The method for producing microcarriers according to claim 11, wherein the biocompatible polymer is selected from the group consisting of proteins, polysaccharides, synthetic polymers and a combination thereof.

13. The method for producing microcarriers according to claim 12, wherein the biocompatible polymer is selected from the group consisting of gelatin, collagen, fibrins, agarose, hyaluronic acid, chitin, alginates, celluloses and gellan gum.

14. The method for producing microcarriers according to claim 13, wherein the Step A comprises introducing a gaseous flow into a continuous fluid to generate bubbles which serve as the dispersed phase.
